(19) United States
(12) Patent Application Publication
(10) Pub. No.: US 2013/0337489 A1
(43) Pub. Date: Dec. 19, 2013

(54) Method for Improved Detection of Microorganisms and the Like

(71) Applicants: Geoffrey N. Roth, Goshen, IN (US); Jonathan N. Roth, Goshen, IN (US)

(72) Inventors: Geoffrey N. Roth, Goshen, IN (US); Jonathan N. Roth, Goshen, IN (US)

(21) Appl. No.: 13/507,259

(22) Filed: Jun. 16, 2012

(51) Int. Cl. C12Q 1/04; C12Q 1/24

(52) U.S. Cl. CPC C12Q 1/04; C12Q 1/24
USPC 435/30; 435/34

(57) ABSTRACT

A method is provided of applying a test sample to a fibrous pad containing growth media which uses unaccelerated dispersion and deposition of the microorganism.

### FIELD OF THE INVENTION

The present invention relates to methods of detecting microbes, bacteria and the like, and, more particularly, to methods of detecting enzyme reactions which are indicative of the presence of bacteria and other microorganisms.

### BACKGROUND OF THE INVENTION

Previously, it has been suggested that membrane filtration methods can be used to detect the presence of microorganisms, such as bacteria, quantify the number of such microorganisms in a given volume of test sample, and even differentiate different types of microorganisms in the test sample. Such methods have included applying growth media to a pad or “barrier destination” substrate, and then forcing test fluid onto the pad via a vacuum or pressure differential applied to the test container. In doing so, the test fluid may be forced first through a micro-pore or nano-pore membrane and then that membrane is applied to the pad, rather than have the pressure differential applied to the pad itself. Once the bacteria are lodged against or trapped on the pad and/or the membrane and positively located or positioned thereon by the vacuum or pressure, the pad or membrane is monitored to detect activity caused by the interaction of the growth media with the particular microorganism for which detection is sought. Typically, the activity detected would be the enzyme reactions associated with the maintenance or growth of the microorganism.

However, some such prior methods have been found to cause damage to the bacteria in the test sample, that damage being sufficient to inhibit growth or detection of the bacteria. This can case either longer periods of time to be used for accurate testing, and/or inaccurate test results, and/or require the use of larger test samples. Further, the prior methods can be more expensive (since they require the use of a vacuum source, and filter funnels, etc.) and require too large a container or test module (thus, preventing a larger number of test samples from being incubated within the same incubator space).

Accordingly, it is an object of the present invention to provide an improved method of detecting microorganisms and the like. Other objects include the provisions of such a detection method which is less expensive to use, more accurate, produces faster results, and produces a greater volume of results within a given time period.

### SUMMARY OF THE INVENTION

These and other objects of the present invention are obtained by the method of applying a test sample to a fibrous pad containing growth media which uses unaccelerated dispersion and deposition of the microorganism. For example, the test sample can be placed onto a membrane and then the membrane placed onto a fibrous pad which has been wetted with an aqueous growth medium. The test sample is then permitted to hydrostatically disperse over the membrane and then wick through the membrane by hydraulic action to lodge the microorganisms at specific locations defined by the fibers of the pad which inhibit further dispersion during the monitoring process. Alternatively, a relatively small amount of the test sample can be placed onto the lower surface of a standard testing dish and the wetted pad then placed immediately over the top of the test sample, thus allowing hydrostatic dispersion under the gravitational force applied by the pad, and wicking of the microorganisms up into the pad by, for example, evaporative fluid dynamic forces.

Other objects, advantages, and novel features of the present invention will become readily apparent to those of skill in the art from the following detailed description and claims.

### DESCRIPTION OF PREFERRED EMBODIMENTS

The present invention in particularly applicable to current testing requirements in the food and beverage industry with respect to detection of bacteria, various pathogens, and other microorganisms and the like. This invention facilitates testing as to the presence or absence of the items to be detected as well as quantitative testing as to the volume of such items and/or differentiation of the various types of items to be detected. This invention is also typically well suited to use. in educational and scientific analysis or detection systems for such items.

In general, standard detection systems involve placing a container, such as a transparent 47 mm diameter dish, having therein a sample of the material to be tested into a visual detection device which allows identification and/or counting (either by the operator manually or by automated equipment) of the microorganisms, as a result of some visual differentiation between the area where the microorganism is located on the dish and the background color of the surface in the dish upon which the sample is mounted. The visual differentiation can, for example, be as a result of an enzyme activity which is uniquely associated with the particular type of bacteria which is sought to be detected. That enzyme activity can, for example, result by creating film of nutrients onto a surface or pad onto which the test sample is mounted in the dish. Any bacteria present in the test sample would be able to then use that nutrient supply to sustain itself and/or grow, such as by an enzyme reaction with the nutrients which would produce a visual effect. That growth may be supported or accelerated by incubation of the test sample, alone or together with a number of other test samples. The standard detections systems may also apply various inhibiting agents or growth agents to the test sample, depending upon the purposes of the particular test.

The present invention presents an improved method for use in such standard detection systems. The method of the present invention allows for faster and more accurate detection of microorganisms and the like by reducing the destruction of the microorganisms prior to detection and by increasing the dispersion of the microorganisms onto the growth medium. As used herein, “growth medium” refers to a substance which can be applied to the locating surface to create a detectable indicia of the presence of the item sought to be detected. In the case of bacteria, for example, the growth medium can be nutrients needed by the particular type of bacteria either for its maintenance and/or growth, where those nutrients produce a visually distinctive indicator as a result of enzyme reaction resulting from the presence of that type of bacteria. Such nutrients are well known in the industry, a wide variety of them being commercially available depending upon the particular bacteria which is sought to be detected. In preferred embodiments of the present invention, the growth media is a water based, or aqueous, solution which is applied to the locating surface prior to testing.

As used herein, “locating surface” refers to a structure upon which the microorganisms or items sought to be detected are ultimately placed in proximity to the growth media. Ideally, the locating surface does not allow the test sample to slide around to move to any significant degree, even when the dish or container is being moved, as between an incubator and a visual detection device. Ideally also, the test sample is fully dispersed over the locating surface such that accurate detection is possible. For example, if a test sample has a high microbial population, insufficiently dispersed test samples would allow one bacteria to mask the location of another bacteria, thus producing an inaccurate “count” of the bacterial presence. Such locating surfaces are well known in the industry, a wide variety of them being commercially available depending upon the particular item which is sought to be detected. In preferred embodiments of the present invention, the locator surface is a fibrous or woven pad which is capable of absorbing an aqueous solution and whose fibers are have sufficient dimension and configuration to entrap or cause attachment thereto of the microorganisms and the like which are sought to be detected. For example, such absorbent pads can be made of a paper material which resembles construction paper.

The inventors hereto have discovered that certain prior methods of detecting microorganisms and the like which have used membrane filtration or pour plate methods are insufficiently accurate, or more costly, or too slow to fully satisfy the needs and desires of the industry. For example, certain prior membrane filtration systems accomplish dispersion of the test sample onto the locating surface or fibrous pad by placing the test sample onto a micro or nano porous membrane and then applying an acceleration force, such as differential air pressure or vacuum, to force the test sample through the membrane. The fibrous pad, which was previously soaked with the appropriate growth media, is immediately adjacent the underside of the membrane, and the test sample passing through the membrane is then in contact with the fibrous pad.

Unfortunately, such prior methods have been found to result in the destruction of the microorganisms, such as certain bacteria, as a result of the acceleration force. Once destroyed, those bacteria cannot react with the growth medium on the fibrous pad, and may give negative presence results or a low bacteria count to the detector. In some cases, prior applications of these methods may have sought to compensate for such effects and/or to improve reliability by increasing the volume of the test sample and/or the incubation time. From the stand point of industrial applications, however, those increases are undesirable and can be unduly costly. Alternatively, other prior methods which are “gentler” to the bacteria may fail to provide appropriate dispersion of the test sample, which can also lead to inaccurate results.

To solve these problems and provide improved testing methods, the present invention teaches that hydrostatic forces and the like which are found using smaller test samples and with wetted surfaces may themselves be sufficient to provide appropriate dispersion and deposition of the microorganisms onto the growth medium and that acceleration forces on the test sample are to be avoided. For example, the method of the present invention includes selecting a fibrous pad conventionally according to the particular item(s) to be detected, and then soaking that fibrous pad in an aqueous solution of the growth medium (the particular growth medium also being selected conventionally according to the particular item(s) to be detected. Then the fibrous pad in placed into the detection container, a 47 mm diameter transparent dish, for example, and a conventional micro or nano porous membrane is then placed onto the top of the fibrous pad. Next, the test sample, preferably in fluid form, is placed onto the top of the membrane. The amount of test sample should not be such that it flows over the edge of the membrane, nor should it completely cover the top surface of the membrane. In some instances the amount of the test sample can be one gram or less.

The test sample is then allowed to disperse laterally over the top surface of the membrane of its own accord, such as by normal forces related to hydraulic surface tension and hydrostatic forces incident to the fluid configuration of the test sample, and wick through the membrane onto the fibrous pad without the use of acceleration forces. Thereafter, the disk is incubated and/or monitored for detection of the desired items in a conventional manner.

In another embodiment of the present invention, the test sample is first placed onto the lower interior surface of the detection container, again preferably in fluid form and in a volume which does not cover the bottom surface of the detection container. Next, the fibrous pad (previously soaked with the growth medium) is placed onto the test sample. That placement itself can allow for appropriate dispersion of the test sample laterally over the lower interior surface of the detection container. The fibrous nature of the pad and the fluidic and/or evaporative forces associated with the interaction of the test sample and the growth medium can allow for upward wicking of the test sample into the fibrous pad, at least sufficient to provide the normal enzyme reactions of bacteria, for example, which thereafter would permit application of conventional detection and counting techniques, through the transparent bottom of the detection container.

Various adaptations of the present invention are anticipated for given applications. For example, depending upon the particular item to be detected, instead of a fibrous pad, a “gum” surface can be used to contain the growth medium and to locate the items to be detected thereon. Also, two embodiments can be combined, such that the membrane barrier is used on one side of a fibrous pad for one test sample, which the other side of the fibrous pad is placed on top of a second test sample. In each case, hydraulic wicking can sufficiently support both appropriate dispersion and deposition of bacteria onto each surface of the pad. Also, the method of the present invention is not limited to the use of dishes as the detection container. In certain applications, embossed films or films capable supporting the test sample, for example, may be sufficient as the detection container.

Accordingly, the present invention has been described herein with respect to certain preferred embodiments, but the spirit and scope of this invention are limited only by the scope of the following claims:

What is claimed is:

1. A method of preparing test samples for item detection, comprising the steps of:
applying a growth medium to a surface which is capable of positively locating item to be detected, and then
applying a test sample to that surface without the use of an acceleration force and by allowing dispersion of the test sample over that surface by hydrostatic and/or hydraulic surface tension forces.

2. The method according to claim 1 wherein the test sample is applied to said surface by the steps of first applying a porous membrane to the top of said surface and then the test sample is applied to the top of that membrane, and then the test sample wicks through that membrane to contact said surface.

3. The method according to claim 2 wherein the growth medium is applied to said surface by the steps of first selecting a fibrous pad suitable for retention of the item to be detected thereon at specific location, then soaking that pad in an aqueous solution containing the growth medium.

4. The method according to claim 3 wherein the fibrous pad in placed into a detection container and the fibrous pad is thereafter monitored for indications of the presence of the item to be detected by virtue of some indicia resulting from the growth medium.

5. The method according to claim 3 wherein the following preliminary steps are used:
a second test sample is placed on the upper surface of a detection container, the volume of that second test sample being insufficient to cover that upper surface,
then, the fibrous pad is placed on top of that second test sample, and
then, the steps set forth in the sequence indicated in claims 1-3 are applied.
